(19) United States
(12) Patent Application Publication
(10) Pub. No.: US 2026/0325409 A1
(43) Pub. Date: Oct. 22, 2026

(54) Aesthetically Improved Dental Ceramics

(71) Applicant: Vita Zahnfabrik H. Rauter GmbH & Co. KG, Bad Säckingen (DE)

(72) Inventors: Christopher Neun, Bad Säckingen (DE); Michael Gödiker, Bad Säckingen (DE)

(21) Appl. No.: 19/648,920

(22) Filed: Apr. 15, 2026

(30) Foreign Application Priority Data
Apr. 17, 2025 (DE) 102025115301.9

(51) Int. Cl. A61K 6/816; A61K 6/813; A61K 6/818; A61K 6/82; A61K 6/822

(52) U.S. Cl. CPC A61K 6/816; A61K 6/813; A61K 6/818; A61K 6/82; A61K 6/822

(57) ABSTRACT

A dental restoration is described, obtainable by a method comprising the steps of:
a) sintering a ceramic material containing titanium dioxide (TiO₂) and/or iron oxide, and subsequently
b) cooling the sintered dental restoration, wherein the cooling is partially or completely controlled.

### CROSS REFERENCE TO RELATED APPLICATIONS

This application claims priority to and the benefit of German Patent Application No. DE 10 2025 115 301.9 filed on Apr. 17, 2025, the entire contents of which are incorporated herein by reference.

### FIELD OF THE INVENTION

The present invention relates to dental restorations obtainable by sintering a ceramic material containing titanium dioxide and/or iron oxide and subsequently cooling the sintered dental restoration in a controlled manner. Furthermore, the invention relates to a method for manufacturing dental restorations.

### BACKGROUND

Oxide ceramic dental restorations have increasingly found their way into dental technology due to their hardness, strength, and resistance properties. In particular, zirconia ceramics have gained importance in the manufacture of dental restorations due to their hardness, good machinability, and controllable translucency properties. The coloring of oxide ceramics, particularly zirconia ceramics, is usually achieved by adding coloring inorganic oxides that are sintered together with the zirconia. To achieve a natural gradient of tooth color, it is advisable to vary the concentration of the coloring oxides layer by layer and adjust it so that the color gradient closely matches that of natural tooth color. However, a problem here is that the coloring metal oxides can influence the sintering behavior of the ceramics. To optimize sintering behavior, titanium dioxide, for example, can be added to the dental ceramic materials. However, it has been found that the addition of titanium dioxide, particularly to dental ceramics containing zirconium dioxide, can lead to unwanted graying of the dental restorations. This graying is often visible to the naked eye and therefore results in a deviation from the desired tooth color. Without being bound to a specific theory, it is hypothesized that titanium suboxides form during or after the sintering process, which could be the cause of the gray haze on the dental restorations. Furthermore, it has been observed that the sintering processes customary in the prior art and the subsequent cooling result in a reduction in color intensity (color saturation) in ceramics containing iron oxide. Typically, dental restorations are cooled to room temperature (20 °C) by natural cooling after the sintering process. This cooling typically occurs by turning off the sintering furnace, resulting in cooling rates of approximately 100 °C/minute in the range close to the sintering temperature.

EP 4137312 A1 describes a method for manufacturing a dental restoration in which an oxide ceramic material is subjected to at least one heat treatment and is cooled, wherein the cooling comprises a first cooling step at a cooling rate T1 and a second cooling step at a cooling rate T2.

### SUMMARY

The present invention is therefore based on the objective of providing dental restorations that solve the aforementioned problems and, in particular, exhibit improved color saturation and reduced graying.

The aforementioned problems are solved by a dental restoration according to the present invention.

The subject matter of the present invention in a first embodiment is a dental restoration obtainable by a method comprising the steps:
a) sintering a ceramic material containing titanium dioxide (TiO₂) and/or iron oxide, and subsequently
b) cooling the sintered dental restoration, wherein the cooling is partially or completely controlled.

It was surprisingly found that controlled cooling, at least in certain regions of the cooling curve-particularly in the higher temperature range, for example above 800 °C, and specifically above 1000 °C—can reduce the graying (gray discoloration) of the dental restoration in ceramic materials containing titanium dioxide. Furthermore, it was found that controlled cooling also leads to an intensification of the color of dental restorations made from ceramic materials containing iron oxide.

The controlled cooling of the sintered dental restorations is typically characterized by the fact that the cooling is slowed down compared to the conventional method in the prior art.

In a preferred embodiment of the present invention, the controlled cooling may comprise gradual cooling and/or stepwise cooling. Typically, controlled cooling is performed over a period of at least 5 minutes, preferably at least 10 minutes, more preferably at least 15 minutes, for example 10 to 200 minutes, specifically 15 to 100 minutes.

In gradual controlled cooling, the cooling rate is significantly slowed down. This can be achieved, for example, via a temperature control system and a cooling program of the sintering furnace. Within the scope of the present invention, controlled cooling comprises a temperature section in which the cooling rate is 1 to 25 °C/min, preferably 2 to 15 °C/min, and in particular 3 to 10 °C/min or 3 to 7 °C/min.

Partially controlled cooling means that cooling occurs only in one or more temperature ranges, i.e., not over the entire temperature range from sintering temperature to room temperature (20 °C). Partially controlled cooling is preferred according to the invention, since an excellent effect is already achieved when the cooling control is carried out in the range of 50 to 800 °C, specifically 80 to 500 °C, below the sintering temperature. Further cooling control in the cooler range is not necessary in some embodiments for economic reasons, due to the greater time required relative to the benefit.

The stepwise cooling can be achieved by maintaining a specific temperature for a specific period of time during the cooling process (isotherm). In a preferred embodiment of the present invention, the controlled cooling is carried out stepwise with at least one isotherm, in particular over a period of more than 5 minutes, in particular of more than 10 minutes, or in a range of 10 to 45 minutes.

It has proven advantageous to perform controlled cooling in a temperature range extending from the sintering temperature up to about 800 °C, specifically in the temperature range from the sintering temperature up to about 1000 °C.

Particularly in the high-temperature range close to the sintering temperature, at least partially controlled cooling is especially effective. In a further embodiment of the present invention, the cooling involves a controlled reduction of the cooling rate, preferably in the temperature range from the sintering temperature to about 800 °C, specifically up to about 1000 °C.

The temperatures typically used for sintering ceramic dental restorations are above 1350 °C, preferably in a range from 1400 °C to 1700 °C, and in particular from 1420 °C to 1650 °C.

In a preferred embodiment, the controlled cooling takes place entirely or partially in a temperature range from 1400 °C to 800 °C, particularly preferably from 1350 °C to 1000 °C.

In a further preferred embodiment of the present invention, the controlled cooling takes place in stages and comprises 2 or more isotherms, each with a holding time or a total holding time of preferably at least 5 minutes, more preferably at least 10 minutes, and particularly preferably between 10 minutes and 120 minutes, for example 15 to 45 minutes.

In a preferred embodiment of the present invention, the controlled cooling comprises two or more isotherms, which are carried out in the temperature range between 1400 °C and 1000 °C with a preferred holding time of 2 to 45 minutes, particularly 5 to 30 minutes.

### DETAILED DESCRIPTION

The dental restorations of the present invention are obtainable by sintering a ceramic material containing titanium dioxide and/or iron oxide, followed by at least partially controlled cooling.

The ceramic material preferably comprises an oxide ceramic material containing titanium dioxide and/or iron oxide.

Ceramic materials containing zirconium dioxide have proven to be particularly suitable.

In one embodiment of the present invention, the ceramic material comprises at least 85 wt % ZrO₂ and/or HfO₂, wherein the weight percentage is based on the total weight of the ceramic material.

In one embodiment, the ceramic material comprises iron oxide, preferably iron oxide and titanium oxide. Iron oxides are known to those skilled in the art of dental technology as inorganic colorants for ceramic dental restorations. In a preferred embodiment of the present invention, the ceramic material comprises iron oxide in an amount of 0.001 to 0.2 wt. %, preferably 0.01 to 0.18 wt. %, wherein the weight percentages are each based on the total weight of the ceramic material.

In a further embodiment of the present invention, the ceramic material contains titanium dioxide. It has been found that titanium dioxide contributes to improving the sintering properties, particularly in the presence of iron oxide. Preferably, the ceramic material contains titanium dioxide in an amount of 0.001 to 1.0 wt. %, preferably 0.002 to 0.8 wt. % or 0.002 to 0.6 wt. %, particularly 0.005 to 0.5 wt. %, wherein the weight percentages (wt. %) are each based on the total weight of the ceramic material.

In a preferred embodiment, the ceramic material comprises yttrium oxide. Typically, the yttrium oxide is present in an amount of 6 to 10 wt. %, particularly preferably 6.5 to 9 wt. %, based on the total weight of the dental restoration.

In a preferred embodiment, the dental restorations of the invention comprise Al₂O₃, preferably in an amount of at least 0.02 wt. %, based on the total weight of the restoration.

It has proven advantageous for the restoration to contain at least 0.02 wt % Al₂O₃, preferably Al₂O₃ in an amount of 0.03 to 0.15 wt %, and in particular Al₂O₃ in an amount of 0.03 to 0.08 wt %. The Al₂O₃ results in improved stability and strength properties of the dental restoration according to the invention.

In a further embodiment, the ceramic material additionally comprises cobalt oxide (Co₃O₄). The cobalt oxide can be used as an additional coloring pigment. Typically, the ceramic material comprises 0.001 to 0.02 wt % cobalt oxide, wherein the weight percentage is based on the ceramic material.

In a further embodiment of the present invention, the ceramic material additionally comprises erbium oxide. Erbium oxide can also be used to adjust the shade of ceramic dental materials, particularly in zirconia-based dental materials. In one embodiment, the ceramic material comprises Er₂ O₃, preferably in an amount of 0 to less than 1 wt. %, in particular from 0.1 to less than 0.9 wt. % or from 0.2 wt. % to 0.8, in particular from 0.25 to 0.7 wt. %, specifically from 0.3 to 0.6 wt. %, in each case based on the total weight of the ceramic material.

Preferably, the ceramic material additionally comprises terbium oxide. The use of terbium oxide has proven to be excellent for both coloring and distortion stability.

Terbium oxide as used in the present invention essentially comprises all oxides of terbium. Preferably, the terbium oxide is selected from the group consisting of Tb₂O₃, Tb₄O₇, Tb₇O₁₂, Tb₁₁O₂₀, TbO₂O₂ and mixtures thereof.

Terbium oxide selected from the group consisting of Tb₄O₇, Tb₇O₁₂, Tb₁₁O₂₀, and any mixtures thereof may be particularly preferred.

Terbium oxide is particularly preferred in the form of Tb₄O₇ or as a mixture of Tb₄O₇ with Tb₇O₁₂ and/or Tb₁₁O₂₀.

Terbium oxides present in mixed valence are also preferred.

Terbium oxide improves the sintering rate and sintering properties, particularly in combination with titanium dioxide. Additionally, it contributes to the coloring of the ceramic material. In one embodiment of the invention, the ceramic material comprises terbium oxide in an amount of 0.001 to 0.25 wt. %, preferably from 0.002 to 0.19 wt. %, in particular from 0.003 to 0.17 wt. %, and specifically from 0.01 to 0.15 wt. %, wherein the weight percentages (wt. %) are each based on the total weight of the ceramic material.

In a further preferred embodiment of the present invention, the restoration exhibits a color gradient and/or a translucency gradient. This is particularly advantageous for replicating the natural color gradient of teeth. It has been shown that color and translucency gradients can be achieved by a layered structure of the ceramic material. For this purpose, pressed bodies comprising two or more color-differentiated ceramic powder layers can be sintered, whereby a color gradient is established with suitable inorganic color pigments and a translucency gradient is established by varying the yttrium oxide content in the layers.

In a preferred embodiment of the present invention, the sintering process involves sintering a pressed body comprising two or more ceramic powder layers of different colors, wherein each powder layer preferably comprises at least 80% by weight of ZrO₂. In preferred embodiments, the pressed body comprises at least 2 powder layers, or at least 3 powder layers, or at least 4 powder layers, specifically 5 powder layers.

In a preferred embodiment, at least one powder layer, preferably at least two powder layers, or at least three powder layers, or at least four powder layers, specifically five powder layers, or each powder layer comprises terbium oxide in an amount of 0.001 to 0.15 wt. %, preferably 0.01 to 0.1 wt. %, in particular 0.02 to 0.08 wt. %, specifically 0.02 to 0.06 wt. % terbium oxide.

It has surprisingly been found that each of the sintered powder layers exhibits a substantially equal change in volume over a temperature range of 25 to 1600 °C, in particular over a temperature range of 50 to 1400 °C, or 900 to 1400 °C, and specifically over a temperature range of 900 to 1350 °C.

For the purposes of the present invention, a substantially equal change in volume means that the difference in the change in volume at a given temperature in the range of 25 to 1600 °C between two layers of the sintered blank according to the invention is at most 1%, preferably at most 0.5%, and in particular at most 0.05%. This results in minimal sintering distortion for the dental restorations of the present invention.

In a further preferred embodiment of the present invention, each powder layer has a different concentration of terbium oxide, erbium oxide, and cobalt oxide.

In a preferred embodiment, each powder layer comprises iron oxide, erbium oxide, and cobalt oxide as coloring metal oxides.

In a further preferred embodiment of the present invention, at least one powder layer, preferably two or more powder layers, in particular all powder layers, is essentially free of elements that produce a fluorescence effect, in particular essentially free of bismuth.

Essentially free of elements producing a fluorescence effect, as used in the present invention, means that less than 0.01 wt. %, preferably less than 0.001 wt. %, or less than 0.0001 wt. %, in each case based on the total weight of the powder layer, is present. In a preferred embodiment, the sintered shaped body is free of further metal oxides that produce a fluorescence effect.

The use of lanthanum oxide in dental restorations can have an adverse effect on the color composition, i.e., the interaction of the coloring metal oxides to be used in the powder compositions employed according to the invention.

In a further preferred embodiment of the present invention, therefore, at least one powder layer, preferably two or more powder layers, and in particular all powder layers, are essentially free of lanthanum oxide.

Essentially free of lanthanum oxide within the meaning of the present invention means that less than 0.004 wt. %, preferably less than 0.001 wt. %, or less than 0.0001 wt. %, in each case based on the total weight of the powder layer, is present. In a preferred embodiment, the restoration is free of lanthanum oxide. Preferably, at least one powder layer, more preferably 2 or more powder layers, and particularly each powder layer, comprises erbium oxide (Er₂O₃) in an amount of 0.1 to 1.0 wt. %, specifically 0.2 to 0.8 wt. %, in each case based on the total weight of the powder layer composition. It has surprisingly been found that erbium oxide can be used as a coloring metal oxide in these amounts without adversely affecting the sintering properties.

In a further preferred embodiment of the present invention, the weight ratio of terbium oxide to cobalt oxide in at least one powder layer, preferably in at least 2 or 3 powder layers, and in particular in each powder layer, is in the range of 80:1 to 5:1, preferably 75:1 to 10:1, and specifically 60:1 to 15:1. Particularly good color and sintering properties were achieved with the aforementioned adjusted weight ratios, especially when using Tb₄O₇.

Each powder layer can preferably be produced by mixing base powders. In a preferred embodiment, each powder layer is produced by mixing 2 or more, preferably 3 or more, and in particular 4 or more base powders. The use of a limited number of base powders in varying amounts for the production of each powder layer offers advantages regarding the compatibility of the individual layers stacked on top of one another and their color gradient in the sintered dental restoration. Suitable base powders can, for example, be obtained from Tosoh.

The sintered dental restorations of the present invention preferably exhibit a gradual color gradient. In one embodiment of the invention, the pressed blank comprises layers of different colors, and the sintering process results in a gradual color gradient.

In a preferred embodiment of the present invention, at least two, preferably all, powder layers have a substantially identical yttrium oxide content.

A substantially identical yttrium oxide content in two or more layers, as defined by the present invention, means that the difference within the layers is no more than 0.1 mol %, preferably no more than 0.05 mol %, and in particular less than 0.01 mol %.

In an alternative embodiment of the present invention, the sintered molded body exhibits a gradation of translucency. Preferably, the restoration according to the invention exhibits a layer-by-layer increase in the amount of yttrium oxide.

In a preferred embodiment, the layered pressed bodies comprise powder layers, each containing at least 0.02 wt % Al₂O₃.

It has been found to be advantageous for the powder layers to each contain at least 0.02 wt % Al₂O₃, preferably 0.03 to 0.15 wt % Al₂O₃, and in particular 0.03 to 0.08 wt % Al₂O₃. The preferred amount of Al₂O₃ in the individual powder layers results in improved stability and strength properties of the molded bodies according to the invention.

In a further aspect of the present invention, at least one, preferably at least 2 or at least 3 of the powder layers, in particular all powder layers, comprise Y₂O₃ and/or Er₂O₃, preferably in an amount of at least 3 wt. %, in particular at least 5 wt. % or at least 6 wt. %, and in particular from 4.5 to 12 wt. %, specifically from 6 to 10 wt. %, based on the total weight of the components of the powder layer.

The Y₂O₃ has a function of stabilizing the zirconium dioxide crystal phases and is not a coloring metal oxide within the meaning of the present invention.

The erbium oxide (Er₂O₃) has a stabilizing function for the ZrO₂ crystal phases and is a coloring metal oxide within the meaning of the present invention.

Preferably, the powder layers comprise zirconium dioxide and/or HfO₂ in an amount of at least 89 wt. %, preferably in an amount of 89 to 98 wt. %, particularly 90 to 96 wt. %, in each case based on the total weight of the components of the powder layer.

In a preferred embodiment, the powder layers comprise zirconium dioxide and hafnium dioxide; more preferably, the amount of hafnium dioxide is 0.1 to 5 wt. %, specifically 0.5 to 2.5 wt. %, based on the total weight of zirconium dioxide and hafnium dioxide.

In a preferred embodiment of the present invention, the pressed body consists of 3 or 4 or, in particular, 5 different ceramic powder layers.

In a further preferred embodiment of the present invention, the pressed body consists of 5 ceramic powder layers, wherein the first powder layer comprises 20 to 30%, preferably 22 to 28%, the second powder layer 10 to 20%, preferably 12 to 18%, the third powder layer 15 to 25%, preferably 17 to 23%, the fourth powder layer 10 to 20%, preferably 12 to 18%, and the fifth powder layer 20 to 30%, preferably 22 to 28%, of the total thickness of the powder layers arranged one above the other, provided that the total thickness adds up to 100%.

In a further preferred embodiment of the present invention, the pressed body consists of 5 ceramic powder layers, wherein the first powder layer comprises 5 to 20%, preferably 6 to 18%, the second powder layer 5 to 20%, preferably 6 to 18%, the third powder layer 10 to 25%, preferably 11 to 20%, the fourth powder layer 10 to 25%, preferably 11 to 20%, and the fifth powder layer 25 to 65%, preferably 30 to 60%, of the total thickness of the powder layers arranged one above the other, provided that the total thickness adds up to 100%.

In one embodiment of the invention, the sintered shaped body is first pre-sintered and machined by subtractive processes and is preferably then final-sintered in a further step.

In a further embodiment of the present invention, at least one powder layer, preferably all powder layers, additionally comprises organic components, preferably in an amount of 3-6 wt. %, particularly in an amount of 4-5 wt. %. Suitable organic components include, in particular, binders and pressing aids that can be easily removed thermally during the debinding step. Suitable binders for zirconium dioxide sintering powders are known to those skilled in the art. These include, for example, polyvinyl alcohol (PVA).

Preferably, the coating powders have a bulk density below 1.2 g/cm³.

It has proven advantageous to use layer powders having an average particle size D₅₀ of 35 μm-85 μm, preferably 40 μm-80 μm, and in particular 50 μm-70 μm or 40-60 μm. The granule powders are measured dry using laser diffraction with a Cilas granulometer.

Typically, the inorganic components of the base powders—i.e., after removal of organic components such as binders, etc.—have a particle size D₅₀ of 0.1 to 1 μm, preferably 0.2 μm to 0.8 μm, and in particular 0.2 μm to 0.7 μm, as measured by laser diffraction. It has been found that the particle sizes make a positive contribution to sintering and, in particular, to the color transitions between the individual powder layers.

The pressed body to be sintered according to the invention can be obtained by arranging 2 or 3, or in particular 4 or 5 or more, ceramic powder layers on top of one another in layers. The layers can be arranged, for example, in a cylindrical container to form discs. Typically, uniaxial compaction of the powder layers can be performed after each layer is applied. This can be done, for example, using a press punch, although this results only in pre-compaction. The uniaxial compaction of the layers perpendicular to the layer surface is preferably performed at a pressure of 10-20 MPa, particularly 12-15 MPa.

In a further preferred embodiment, the ceramic powder layers arranged one on top of the other are compacted to form a compacted body by first compacting uniaxially and perpendicular to the layer surface, preferably to form a pre-compacted body with a density below 2.8 g/cm³, preferably with a density in the range of 2.5-2.7 g/cm³, e.g., 2.65 g/cm³. The uniaxial precompaction can lead to better and more intimate mixing and thus a uniform transition between the layers.

In another preferred embodiment, the compaction to produce the molded body is carried out isostatically, wherein the isostatic compaction is preferably performed following uniaxial precompaction, resulting in a molded body with a density below 3.4 g/cm³, in particular a density of 2.80-3.3 g/cm³, specifically a density of 2.85-3.25 g/cm³. The isostatic pressing is preferably carried out after all layers of the molded body have been arranged. Suitable pressures for isostatic pressing are typically in the range of 500-10,000 bar, preferably in the range of 800-8,000 bar, e.g., 1,000-7,000 bar or 1,000-3,000 bar.

The thicknesses of the individual powder layers of the molded body may vary. In a preferred embodiment, at least two of the ceramic powder layers differ in thickness. Preferably, at least two of the ceramic powder layers of the molded body have a thickness difference of at least 5%. Typically, the pressed bodies may be in the form of cylindrical, circular discs with diameters in the range of 50 to 200 mm, for example 75 to 150 mm. The total thickness of the cylindrical discs can, for example, be in the range of 8 to 40 mm, preferably 10 to 30 mm, and specifically 13 to 25 mm. These dimensions refer to the pressed body in its unsintered state.

With regard to color design and subsequent processing, it has proven advantageous for at least one of the outer ceramic powder layers, preferably both outer ceramic powder layers of the pressed body, to have a greater thickness than a ceramic powder layer located between the outer ceramic powder layers. In particular, when using the ceramic molded bodies produced according to the invention for the manufacture of dental restorations, the layer structure described above with at least one thicker outer layer has proven advantageous, as this provides a suitable structure for machining in CAD/CAM systems or other subtractive machining processes.

In a particularly preferred embodiment of the present invention, the pressed body comprises five ceramic powder layers, wherein the first powder layer comprises 20-30%, preferably 22-28%, the second powder layer comprises 10-20%, preferably 12-18%, the third powder layer comprises 15-25%, preferably 17-23%, the fourth powder layer 10-20%, preferably 12-18%, and the fifth powder layer 20-30%, preferably 22-28%, of the total thickness of the powder layers arranged one above the other, provided that the total thickness adds up to 100%.

In a further embodiment of the present invention, pre-sintering is carried out at a temperature in the range of 950-1100 °C, preferably 980-1050 °C, to form a pre-sintered ceramic body. Typically, the pre-sintering is carried out over a period of time sufficient to remove the existing binders and to impart sufficient strength to the pressed body for machining by subtractive processes. The pre-sintered and debound pressed bodies are referred to as pre-sintered bodies.

In one embodiment, the pre-sintering for producing the pre-sintered body takes place over a period of more than 30 minutes, preferably more than 1 hour, in particular more than 20 hours or more than 50 hours, for example 60 to 200 hours or 70 to 150 hours.

Particularly for the production of ceramic dental restorations, it is advantageous for the pre-sintered ceramic pre-sintered body to be machined using subtractive processes and preferably subsequently final-sintered in a further step. When using subtractive processes, sintering shrinkage is typically factored into the calculations.

Sintering according to the invention is typically performed at temperatures above 1350 °C, preferably above 1400 °C, specifically in the range of 1420 °C to 1600 °C or 1450 °C to 1590 °C or 1480 °C to 1580 °C.

The sintering duration for the sintering according to the invention is typically over a period of more than 4 minutes, preferably more than 5 minutes, particularly in the range of 5 to 120 minutes.

In a further preferred embodiment of the present invention, the dental restoration of the invention is obtainable by a method comprising the steps of:
i) providing 2 or 3 or 4 or 5 or more, preferably color-differentiated, ceramic powder layers arranged in layers one above the other,
ii) pressing the ceramic powder layers arranged in layers one on top of the other to form a pressed body, and
iii) pre-sintering the molded body obtained in step ii) to form a ceramic blank,
iv) subtractive machining of the pre-sintered body to form a dental restoration,
   a) Sintering the ceramic material in a temperature range above 1350 °C and subsequently
   b) cooling the sintered dental restoration, wherein the cooling is partially or fully controlled.

In a further embodiment, the ceramic powder layers each have a composition different from one another, and wherein each powder layer comprises, preferably at least 85 wt % ZrO₂ and/or HfO₂, Y₂O₃, cobalt oxide, TiO₂, and

optionally Er₂O₃ and/or terbium oxide,
wherein the weight percentages are each based on the powder layer.

In a further embodiment of the invention, the pressed body comprises 2 or more, preferably 3 or more, more preferably 4 or more, specifically 5 ceramic powder layers, wherein each powder layer
a) at least 85 wt. % ZrO₂ and/or HfO₂,
b) 6 to 10 wt. % Y₂O₃,
c) 0 to 0.25 wt. % terbium oxide
d) 0.002 to 1.0 wt. % TiO₂ and/or
d1) 0.001 to 0.2 wt. % iron oxide; and
e) optionally 0 to 0.7 wt. % Er₂O₃; and
f) optionally cobalt oxide,
   wherein the weight percentages are each based on the powder layer.

The base powder preferably comprises yttrium oxide in an amount of 5 to 8 wt. %, preferably 6 to 7 wt. %, terbium oxide in an amount of 0.1 to 0.4 wt. %, preferably 0.1 to 0.3 wt. %, and zirconium dioxide in an amount greater than 80 wt. %, preferably from 82 wt % to 94 wt %, specifically 84 wt % to 90 wt %, in each case based on the total weight of the base powder.

A further object of the present invention is a method for manufacturing a dental restoration comprising the steps of:
i) providing 2 or 3 or 4 or 5 or more, preferably color-differentiated, ceramic powder layers arranged in layers one above the other,
ii) pressing the ceramic powder layers arranged one on top of the other to form a pressed body, and
iii) pre-sintering the molded body obtained in step ii) to form a ceramic blank,
iv) subtractive machining of the pre-sintered body to form a dental restoration,
   a) Sintering the ceramic material in a temperature range above 1350 °C and subsequently
   b) cooling the sintered dental restoration, wherein the cooling is partially or fully controlled.

Preferred embodiments of the method according to the invention have already been described above.

The dental restorations of the invention may take any form, for example as crowns, bridges, onlays, or inlays.

### Examples 1 through 3 and Comparative Example 1

Table 1 shows the compositions of 5 layers of a pressed body.

The D₅₀ particle size of the powders is in the range of 40-80 μm. The inorganic components of the base powders have a D₅₀ particle size of 0.2 to 0.7 μm. The weight values for each layer refer to the total weight of the powder layer.

The pressed bodies are intended for use in the manufacture of dental restorations, so the layer compositions are designed according to their position in the tooth. The composition of each powder layer is achieved by homogeneously mixing the specified quantities. The powders are then placed layer by layer into a cylindrical mold with a diameter of 100 mm, and a layer thickness of 18 mm is set. The powder layers are pre-pressed uniaxially at a pressure of 13 MPa perpendicular to the layer surface and subsequently isostatically pressed at a pressure of 2000 bar. This is followed by debinding and pre-sintering at approximately 1000 °C in a production furnace (Nabertherm N450) over a period of approximately 100 hours. The pre-sintered bodies obtained in this way are milled into dental bridges as dental restorations using CAD/CAM systems.

TABLE 1
Layer compositions:

| Layer | Component | [wt. %] |
|---|---|---|
| 1 (incisal) | Y₂O₃ | 7.99 |
| | TiO₂ | 0.12 |
| | Al₂O₃ | 0.028 |
| | Fe₂O₃ | 0.085 |
| | Co₃O₄ | 0.003 |
| | Er₂O₃ | 0.522 |
| | Organic binder | 4 |
| | ZrO₂ | to 100 |
| 2(incisal) | Y₂O₃ | 7.45 |
| | TiO₂ | 0.30 |
| | Al₂O₃ | 0.035 |
| | Fe₂O₃ | 0.12 |
| | Co₃O₄ | 0.0025 |
| | Er₂O₃ | 0.626 |
| | Organic binder | 4 |
| | ZrO₂ | to 100 |
| 3(Dentin) | Y₂ O₃ | 6.90 |
| | TiO₂ | 0.55 |
| | Al₂O₃ | 0.043 |
| | Fe₂O₃ | 0.15 |
| | Co₃O₄ | 0.0024 |
| | Er₂O₃ | 0.79 |
| | Organic binder | 4 |
| | ZrO₂ | to 100 |
| 4(Dentin) | Y₂O₃ | 6.39 |
| | TiO₂ | 0.75 |
| | Al₂O₃ | 0.050 |
| | Fe₂O₃ | 0.162 |
| | Co₃O₄ | 0.0032 |
| | Er₂O₃ | 0.93 |
| | Organic binder | 4 |
| | ZrO₂ | to 100 |
| 5(cervical) | Y₂O₃ | 6.32 |
| | TiO₂ | 0.74 |
| | Al₂O₃ | 0.050 |
| | Fe₂O₃ | 0.160 |
| | Co₃O₄ | 0.004 |
| | Er₂O₃ | 1.044 |
| | Organic binder | 4 |
| | ZrO₂ | to 100 |

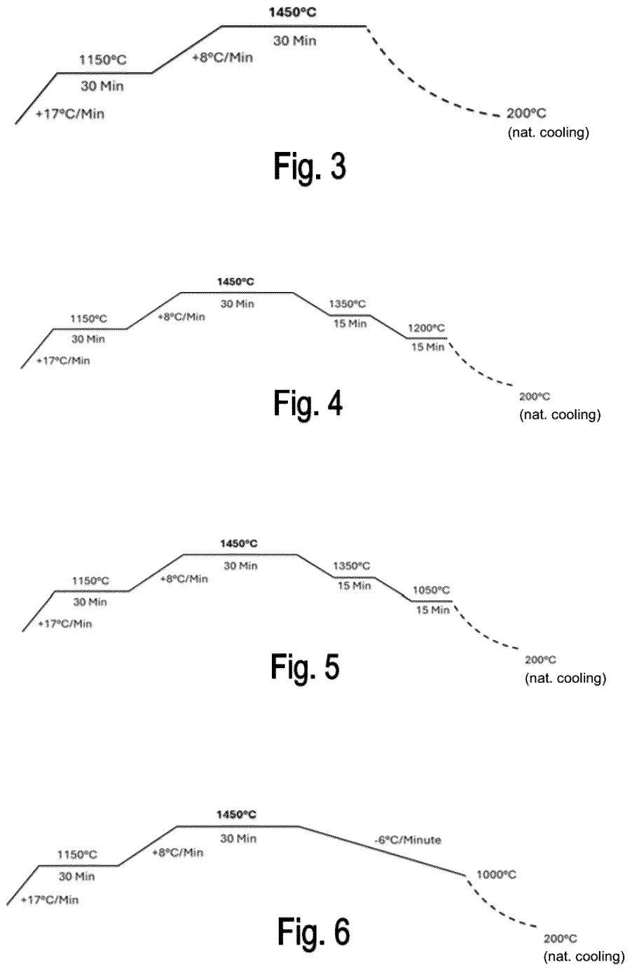

These pre-sintered and machined blanks are then sintered and cooled in a dental furnace (VITA ZYRCOMAT 6100 MS) according to the temperature profiles shown in FIGS. 3 through 6.

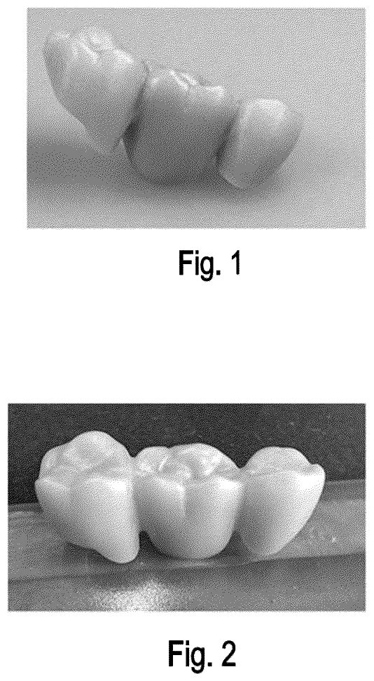

FIG. 3 shows the sintering of the processed white ceramic at 1450 °C for 30 minutes, followed by uncontrolled cooling (comparative example 1). The resulting dental bridge is shown in FIG. 1. A distinct gray discoloration is visually apparent, particularly in the central region.

FIG. 4 shows the sintering of the processed white ceramic at 1450 °C for 30 minutes followed by controlled cooling (Example 1). During cooling, an isothermal stage at 1350 °C and an isothermal stage at 1200 °C were maintained for 15 minutes each. The resulting dental bridge is shown in FIG. 2. No graying was visually detectable.

FIG. 5 shows the sintering of the processed white ceramic at 1450 °C for 30 minutes followed by controlled cooling (Example 2). During cooling, an isothermal curve at 1350 °C and an isothermal curve at 1050 °C were maintained for 15 minutes each. The resulting dental bridge showed no visible discoloration.

FIG. 6 shows the sintering of the processed white ceramic at 1450 °C for 30 minutes followed by controlled cooling (Example 3). During cooling, a gradual cooling process was performed at a cooling rate of 6 °C per minute from the sintering temperature down to 1000 °C. The resulting dental bridge showed no visible graying.

TABLE 2
Measurement of CIE L* values of dental restorations using
a colorimeter (dental bridge measured at the pontic)

| | L* | a* | b* | ΔE |
|---|---|---|---|---|
| Comparative example 1 | 60.8 | 3.7 | 40.5 | — |
| Example 1 | 61.7 | 4.0 | 43.5 | 3.1 |
| Example 2 | 63.0 | 4.0 | 44.4 | 4.5 |
| Example 3 | 61.3 | 4.9 | 45.5 | 5.2 |

ΔE represents the difference between the comparative example and Examples 1 through 3 of the invention.

### Examples 4 through 6 and Comparative Example 2

Table 3 shows the compositions of 5 layers of a pressed body.

The D₅₀ particle size of the powders is in the range of 40-80 μm. The inorganic components of the base powders have a D₅₀ particle size of 0.2 to 0.7 μm. The weight values for each layer refer to the total weight of the powder layer.

The pressed bodies are intended for use in the manufacture of dental restorations, so the layer compositions are designed according to their position in the tooth. The composition of each powder layer is achieved by homogeneously mixing the specified quantities. The powders are then placed layer by layer into a cylindrical mold with a diameter of 100 mm, and a layer thickness of 18 mm is set. The powder layers are pre-pressed uniaxially at a pressure of 13 MPa perpendicular to the layer surface and subsequently isostatically pressed at a pressure of 2000 bar. This is followed by debinding and pre-sintering at approximately 1000 °C in a production furnace (Nabertherm N450) over a period of approximately 100 hours. The pre-sintered bodies obtained in this way are milled into crowns as dental restorations using CAD/CAM systems.

TABLE 3
Layer compositions:

| Layer | Component | [wt. %] |
|---|---|---|
| 1 (incisal) | Y₂ O₃ | 7.93 |
| | TiO₂ | 0.11 |
| | Al₂O₃ | 0.028 |
| | Fe₂O₃ | 0.115 |
| | Co₃O₄ | 0.004 |
| | Er₂O₃ | 0.522 |
| | Organic binder | 4 |
| | ZrO₂ | to 100 |
| 2(incisal) | Y₂O₃ | 7.38 |
| | TiO₂ | 0.32 |
| | Al₂O₃ | 0.035 |
| | Fe₂O₃ | 0.138 |
| | Co₃O₄ | 0.004 |
| | Er₂O₃ | 0.696 |
| | Organic binder | 4 |
| | ZrO₂ | to 100 |
| 3(Dentin) | Y₂ O₃ | 6.84 |
| | TiO₂ | 0.54 |
| | Al₂O₃ | 0.043 |
| | Fe₂O₃ | 0.161 |
| | Co₃O₄ | 0.004 |
| | Er₂O₃ | 0.870 |
| | Organic binder | 4 |
| | ZrO₂ | to 100 |
| 4(Dentin) | Y₂ O₃ | 6.39 |
| | TiO₂ | 0.74 |
| | Al₂O₃ | 0.050 |
| | Fe₂O₃ | 0.160 |
| | Co₃O₄ | 0.005 |
| | Er₂O₃ | 0.928 |
| | Organic binder | 4 |
| | ZrO₂ | to 100 |
| 5(cervical) | Y₂O₃ | 6.42 |
| | TiO₂ | 0.72 |
| | Al₂O₃ | 0.050 |
| | Fe₂O₃ | 0.154 |
| | Co₃O₄ | 0.006 |
| | Er₂O₃ | 0.870 |
| | Organic binder | 4 |
| | ZrO₂ | to 100 |

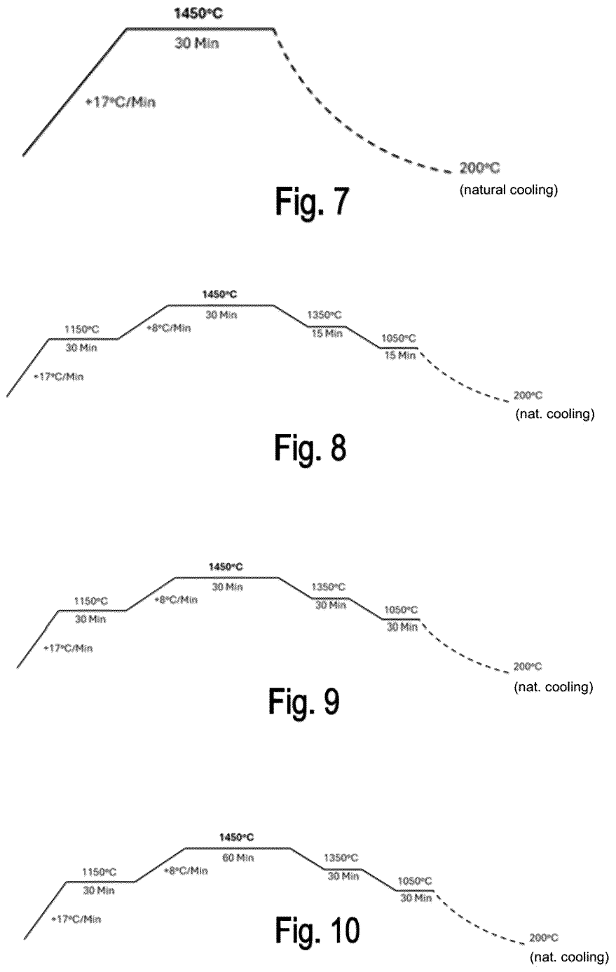

These pre-sintered and machined pre-sintered bodies are then sintered and cooled in a dental furnace (VITA ZYRCOMAT 6100 MS) according to the temperature profiles shown in FIGS. 7 through 10.

FIG. 7 shows the sintering of the machined blank with sintering at 1450 °C for 30 minutes followed by uncontrolled cooling (comparison example 2). A distinct gray discoloration of the crown is visible.

FIG. 8 shows the sintering of the processed blank at 1450 °C for 30 minutes followed by controlled cooling (Example 4). During cooling, an isothermal stage at 1350 °C and an isothermal stage at 1050 °C were maintained for 15 minutes each. The resulting crown showed no visible graying.

FIG. 9 shows the sintering of the processed white metal with sintering at 1450 °C for 30 minutes followed by controlled cooling (Example 4). During cooling, an isothermal curve at 1350 °C and an isothermal curve at 1050 °C were maintained for 30 minutes each. The resulting crown showed no visible graying.

FIG. 10 shows the sintering of the processed white metal with sintering at 1450 °C for 60 minutes followed by controlled cooling (Example 4). During cooling, an isothermal curve at 1350 °C and an isothermal curve at 1050 °C were maintained for 30 minutes each. The resulting crown showed no visible graying.

TABLE 4
Measurement of CIE L* values of dental restorations using
a colorimeter (crown measured at its thickest point)

| | L> | a* | b* | ΔE |
|---|---|---|---|---|
| Comparative example 2 | 67.4 | 2.7 | 40.4 | — |
| Example 4 | 69.5 | 3.3 | 43.5 | 3.8 |
| Example 5 | 68.4 | 3.5 | 42.6 | 2.5 |
| Example 6 | 68.1 | 3.6 | 43.6 | 3.4 |

ΔE represents the difference between the comparative example and Examples 4 through 6 of the invention.

### Examples 7 and Comparative Example 3

Table 5 shows the compositions of 5 layers of a molded body.

The D₅₀ particle size of the powders ranges from 40 to 80 μm. The inorganic components of the base powders have a D₅₀ particle size of 0.2 to 0.7 μm. The weight specifications for each layer refer to the total weight of the powder layer.

The pressed bodies are intended for use in the manufacture of dental restorations, so the layer compositions are designed according to their position in the tooth. The composition of each powder layer is achieved by homogeneously mixing the specified quantities. The powders are then placed layer by layer into a cylindrical mold with a diameter of 100 mm, and a layer thickness of 18 mm is set. The powder layers are pre-pressed uniaxially at a pressure of 13 MPa perpendicular to the layer surface and subsequently isostatically pressed at a pressure of 2000 bar. This is followed by debinding and pre-sintering at approximately 1000 °C in a production furnace (Nabertherm N450) over a period of approximately 100 hours.

The pre-sintered bodies obtained in this way are milled into dental bridges as dental restorations using CAD/CAM systems.

TABLE 5
Layer compositions:

| Layer | Component | [wt. %] |
|---|---|---|
| 1 (incisal) | Y₂ O₃ | 6.53 |
| | Al₂ O₃ | 0.05 |
| | Fe₂ O₃ | 0.101 |
| | Co₃ O₄ | 0.010 |
| | Er₂ O₃ | 0.092 |
| | Organic binder | 4 |
| | ZrO₂ | to 100 |
| 2 | Y₂O₃ | 6.49 |
| | Al₂O₃ | 0.05 |
| | Fe₂O₃ | 0.117 |
| | Co₃O₄ | 0.010 |
| | Er₂O₃ | 0.138 |
| | Organic binder | 4 |
| | ZrO₂ | to 100 |
| 3 | Y₂O₃ | 6.50 |
| | Al₂O₃ | 0.05 |
| | Fe₂O₃ | 0.130 |
| | Co₃O₄ | 0.009 |
| | Er₂O₃ | 0.184 |
| | Organic binder | 4 |
| | ZrO₂ | ad 100 |
| 4 | Y₂O₃ | 6.41 |
| | Al₂O₃ | 0.05 |
| | Fe₂O₃ | 0.145 |
| | Co₃O₄ | 0.010 |
| | Er₂O₃ | 0.230 |
| | Organic binder | 4 |
| | ZrO₂ | to 100 |
| 5 (cervical) | Y₂O₃ | 6.33 |
| | Al₂O₃ | 0.05 |
| | Fe₂O₃ | 0.149 |
| | Co₃O₄ | 0.012 |
| | Er₂O₃ | 0.276 |
| | Organic binder | 4 |
| | ZrO₂ | up to 100 |

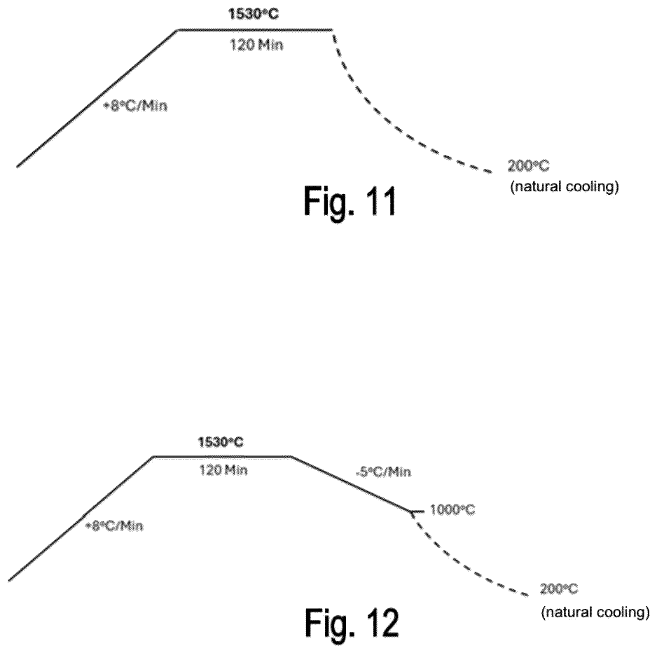

These pre-sintered and machined pre-sintered bodies are then sintered and cooled in a dental furnace (VITA ZYRCOMAT 6100 MS) according to the temperature profiles shown in FIGS. 11 and 12.

FIG. 11 shows the sintering of the machined blank at 1530 °C for 120 minutes followed by uncontrolled cooling (comparative example 3). A reduced color saturation of the bridge is evident.

FIG. 12 shows the sintering of the processed blank at 1530 °C for 120 minutes followed by controlled cooling (Example 7). During cooling, a gradual cooling process was performed at a cooling rate of 5 °C from 1530 °C to 1000 °C. The resulting bridge exhibited improved color saturation visually.

TABLE 6
Measurement of CIE L* values of dental restorations using
a colorimeter (dental bridge measured at the pontic)

| | LA | a* | b* | ΔE |
|---|---|---|---|---|
| Comparative example 3 | 73.3 | 5.2 | 37.3 | — |
| Example 7 | 68.5 | 5.8 | 41.1 | 6.2 |

ΔE represents the difference between the comparative example and Example 7 of the invention. Thus, improved color saturation was measured for Example 7 of the invention.

1. A dental restoration obtainable by a method comprising the steps of:
a) sintering a ceramic material containing titanium dioxide (TiO₂) and/or iron oxide, and subsequently
b) cooling the sintered dental restoration, wherein the cooling is partially or completely controlled, wherein the controlled cooling comprises a temperature section that takes place at a cooling rate of 1 to 25 °C/min; or
the controlled cooling is carried out in stages and 2 or more isothermal stages with a holding time of at least 5 minutes.

2. A dental restoration according to claim 1, characterized in that the sintering takes place in a temperature range above 1350 °C.

3. A dental restoration according to claim 1, characterized in that the controlled cooling takes place in an upper temperature range from the sintering temperature up to about 800 °C.

4. A dental restoration according to claim 1, characterized in that the controlled cooling comprises a temperature range in which the cooling rate is 2 to 15 °C/min.

5. A dental restoration according to claim 1, characterized in that the controlled cooling is performed in stages, wherein at least one isotherm in the temperature range above 800 °C is maintained in particular over a period of more than 5 minutes.

6. A dental restoration according to claim 5, characterized in that the controlled cooling takes place in stages and comprises 2 or more isotherms with a holding time of at least 10 minutes.

7. A dental restoration according to claim 1, characterized in that the controlled cooling takes place wholly or partially in a temperature range of 1400 to 800 °C.

8. A dental restoration according to claim 1, characterized in that the ceramic material comprises at least 85 wt % ZrO₂ and/or HfO₂, wherein the weight percentage is based on the total weight of the ceramic material.

9. A dental restoration according to claim 1, characterized in that the ceramic material comprises iron oxide in an amount of 0.001 to 0.2 wt. %.

10. A dental restoration according to claim 1, characterized in that the restoration exhibits a color gradient and/or a translucency gradient.

11. A dental restoration according to claim 1, characterized in that the ceramic material comprises Er₂O₃ in an amount of 0 to less than 1 wt. %, based on the total weight of the ceramic material, and/or
the ceramic material contains terbium oxide in an amount of 0.001 to 0.25 wt. %, based on the total weight of the ceramic material.

12. A dental restoration according to claim 1, characterized in that the ceramic material comprises titanium dioxide in an amount of 0.001 to 1.0 wt. %, based on the total weight of the ceramic material.

13. A dental restoration according to claim 1, obtainable by a method comprising the steps of:
i) providing 2 or 3 or 4 or 5 or more ceramic powder layers arranged in layers one above the other,
ii) pressing the ceramic powder layers arranged in layers one on top of the other to form a pressed body, and
iii) pre-sintering the pressed body obtained in step ii) to form a ceramic pre-sintered body,
iv) subtractive machining of the pre-sintered body to form a dental restoration,
a) sintering the ceramic material in a temperature range above 1350 °C and, subsequently,
b) cooling the sintered dental restoration, wherein the cooling is partially or completely controlled.

14. Dental restoration according to claim 13, characterized in that the ceramic powder layers each have a composition different from one another, and wherein each powder layer comprises
at least 85 wt % ZrO₂ and/or HfO₂, Y₂O₃, terbium oxide, TiO₂, and Er₂O₃,
where the weight values are based on the powder layer.

15. A dental restoration according to claim 13, characterized in that the pressed body comprises 2 or more ceramic powder layers, wherein each powder layer
a) at least 85 wt % ZrO₂ and/or HfO₂,
b) 6 to 10 wt % Y₂O₃,
c) 0 to 0.25 wt % terbium oxide
d) 0.002 to 1.0 wt % TiO₂ and/or
d1) 0.001 to 0.2 wt % iron oxide; and
e) 0 to 0.7 wt % Er₂O₃; and
f) cobalt oxide,
wherein the weight percentages are each based on the powder layer.
